(19) United States
(12) Patent Application Publication
(10) Pub. No.: US 2024/0207584 A1
(43) Pub. Date: Jun. 27, 2024

(54) Intravenous Catheter Insertion Device and Method of Use

(71) Applicant: Vascular Pathways, Inc., Salt Lake City, UT (US)

(72) Inventors: Amir Belson, Los Altos, CA (US); Gregory W. Hall, Los Gatos, CA (US); Scott A. Daniel, Hayward, CA (US); Robert Brommer, Fremont, CA (US)

(21) Appl. No.: 18/601,899

(22) Filed: Mar. 11, 2024

Related U.S. Application Data
(63) Continuation of application No. 16/867,349, filed on May 5, 2020, now Pat. No. 11,925,778.
Application No. 16/867,349 is a continuation of application No. 16/292,076, filed on Mar. 4, 2019, now Pat. No. 11,020,571.
Application No. 16/292,076 is a continuation of application No. 14/866,441, filed on Sep. 25, 2015, now Pat. No. 10,220,191.
Application No. 14/866,441 is a continuation of application No. 11/577,491, filed on Aug. 20, 2008, now Pat. No. 9,162,037.
(60) Provisional application No. 60/697,333, filed on Jul. 6, 2005.

(51) Int. Cl. A61M 25/09; A61M 5/32; A61M 25/01; A61M 25/06

(52) U.S. Cl. CPC A61M 25/09041; A61M 25/0606; A61M 25/0631; A61M 25/09; A61M 5/3232; A61M 5/3234; A61M 5/3243; A61M 5/3257; A61M 25/01; A61M 25/065; A61M 2025/09133

(57) ABSTRACT

A method for accessing a blood vessel using a catheter insertion device. The catheter insertion device includes a housing, a needle, a catheter, a guidewire, and a thumbwheel. The thumbwheel is rotatably fastened at a front end of the housing and accessible through an opening in the housing. The guidewire is coupled to the thumbwheel such that an axis of rotation of the thumbwheel is perpendicular to the guidewire and rotation of the thumbwheel advances the guidewire through the lumen of the needle. The method includes preparing the catheter insertion device, inserting a tip of the needle and distal end of the catheter into a blood vessel, advancing the guidewire out of the lumen of the needle into the blood vessel by holding the housing and rotating the thumbwheel, furthering the distal end of the catheter over the guidewire, and removing the needle and the guidewire from the catheter.

### CROSS REFERENCE TO RELATED APPLICATIONS

This application is a continuation of U.S. patent application Ser. No. 16/867,349, filed May 5, 2020, now U.S. Pat. No. 11,925,778, which is a continuation of U.S. patent application Ser. No. 16/292,076, filed Mar. 4, 2019, now U.S. Pat. No. 11,020,571, which is a continuation of U.S. patent application Ser. No. 14/866,441, filed Sep. 25, 2015, now U.S. Pat. No. 10,220,191, which is a continuation of U.S. patent application Ser. No. 11/577,491, filed Aug. 20, 2008, now U.S. Pat. No. 9,162,037, which is a U.S. national stage application under 35 U.S.C. § 371 of International Application No. PCT/US2006/026671, filed Jul. 6, 2006, which claims the benefit of priority to U.S. Provisional Application No. 60/697,333, filed Jul. 6, 2005, each of which is incorporated by reference in its entirety into this application.

### FIELD OF THE INVENTION

The present invention relates to devices and methods for insertion and placement of an intravenous catheter into a vein or artery of a patient. The devices and methods of the invention facilitate safe placement of the catheter into the patient's vein or artery, which is of particular importance in the case of small, tortuous, collapsed, fragile, and/or difficult to locate vessels. The devices and methods also provide protection against accidental punctures and/or contamination by the needle after placement of the intravenous catheter.

### BACKGROUND OF THE INVENTION

The following patents and publications describe prior intravenous catheter insertion devices and/or safety devices for syringes and needles: EP Patent No. 0 515 710 B1 to Haining, titled, “Intravenous catheter and insertion device”; U.S. Pat. No. 5,019,049 to Haining, titled, “Intravenous catheter and insertion device”; U.S. Pat. No. 5,176,650 to Haining, titled, “Intravenous catheter and insertion device”; EP Patent No. 0 567 321 B1 to Chang, titled, “Intravenous catheter with needle guard”; EP Patent No. 0 652 020 B1 to Mahurkar, titled, “Retractable hypodermic needle assembly”; EP Patent No. 0 910 988 B1 to Mahurkar, titled, “Blood sample collection assembly”; U.S. Pat. No. 5,891,105 to Mahurkar, titled, “Hypodermic needle assembly”; U.S. Pat. No. 3,572,334 to DeWitt, titled, “Intravenous catheter placement unit”; EP Publication No. 0 750 916 to van Heugten, titled, “Protective needle cover containment”; EP Patent No. 0 942 761 B1 to Botich, titled, “Medical device with retractable needle”; EP Patent No. 1 075 850 B1 to Botich, titled, “Apparatus for intravenous catheter insertion”; U.S. Pat. No. 5,800,395 to Botich et al, titled, “Medical device with retractable needle”; U.S. Pat. No. 6,436,070 to Botich et al, titled, “Catheter insertion device with retractable needle”; U.S. Patent Publication No. 2003/060760 to Botich et al, titled, “Catheter insertion device with retractable needle”; WO 2000/012160 to Botich et al, titled, “Fluid infusion device with retractable needle”; WO 1996/032981 to Botich et al, titled, “Safety stylet for intravenous catheter insertion”; WO 1998/024494 to Botich et al, titled, “Medical device with retractable needle”; EP Patent No. 1 457 229 B1 to Shue, titled, “Intravenous catheter inserting device”; U.S. Patent Publication No. 2004/106903 to Shue, titled, “Intravenous catheter inserting device”; U.S. Pat. No. 3,592,192 to Harautuneian, titled, “Intravenous catheter apparatus with catheter telescoped on outside of puncturing cannula”; U.S. Pat. No. 3,610,240 to Harautuneian, titled, “Intravenous catheter apparatus with catheter telescoped inside puncturing cannula”; U.S. Pat. No. 4,037,600 to Poncy et al, titled, “Catheter placement system”; U.S. Pat. No. 4,292,970 to Hession, titled, “Apparatus for intravenous catheter starter”; U.S. Pat. No. 4,834,718 to McDonald, titled, “Safety needle apparatus”; U.S. Pat. No. 4,944,725 to McDonald, titled, “Safety needle apparatus”; U.S. Pat. No. 4,909,793 to Vining et al, titled, “Intravenous catheter apparatus with retractable stylet”; U.S. Pat. No. 4,944,728 to Carrell et al, titled, “Intravenous catheter placement device”; U.S. Pat. No. 4,966,589 to Kaufman, titled, “Intravenous catheter placement device”; U.S. Pat. No. 5,007,901 to Shields, titled, “Intravenous catheter insertion device”; U.S. Pat. No. 5,562,629 to Haughton et al, titled, “Catheter placement system utilizing a handle, a sharp, and a releasable retainer mechanism providing retraction of the sharp upon disengagement of the catheter from the handle”; U.S. Pat. No. 5,562,634 to Flumene et al, titled, “Intravenous catheter with automatically retracting needle-guide”; U.S. Pat. No. 5,573,510 to Isaacson titled, “Safety intravenous catheter assembly with automatically retractable needle”; U.S. Pat. No. 6,056,726 to Isaacson, titled, “Self-contained safety intravenous catheter insertion device”; WO 1995/023003 to Isaacson, titled, “Self-contained safety intravenous catheter insertion device”; U.S. Pat. No. 5,891,098 to Huang, titled, “Safety intravenous catheter”; U.S. Pat. No. 5,941,854 to Bhitiyakul, titled, “Intravenous catheter”; U.S. Pat. No. 5,997,507 to Dysarz titled, “Biased spring hard needle retractable IV catheter”; U.S. Pat. No. 6,193,690 to Dysarz titled, “Inclined plane latching device for an IV catheter”; U.S. Pat. No. 6,221,047 to Greene et al, titled, “Safety intravenous catheter assembly and method for use with a needle”; U.S. Pat. No. 6,689,102 to Greene et al, titled, “Safety intravenous catheter assembly”; U.S. Pat. No. 6,695,814 to Greene et al, titled, “Safety intravenous catheter assembly and method for use with a needle”; U.S. Patent Publication No. 2001/014786 to Greene et al, titled, “Safety intravenous catheter assembly and method for use with a needle”; U.S. Patent Publication No. 2002/165497 to Greene et al, titled, “Safety intravenous catheter assembly”; WO 2000/006226 to Greene et al, titled, “Safety intravenous catheter assembly and method for use with a needle”; U.S. Pat. No. 6,322,537 to Chang, titled, “Safety intravenous catheter”; U.S. Pat. No. 6,620,136 to Pressly, Sr. et al, titled, “Retractable IV catheter placement device”; WO 2000/047256 to Pressly, Sr. et al, titled, “Retractable IV catheter placement device”; U.S. Pat. No. 6,730,062 to Hoffman et al, titled, “Safety catheter with non-removable retractable needle”; U.S. Patent Publication No. 2003/073956 to Hoffman et al, titled, “Safety catheter with non-removable retractable needle”; U.S. Patent Publication No. 2004/267204 to Brustowicz, titled, “On-demand needle retaining and locking mechanism for use in intravenous catheter assemblies”; WO 2003/043686 to Garcia Andreo, titled, “Flow regulating/autovalve intravenous catheter”; WO 1992/022344 to Sircom, titled, “Needle guard for intravenous catheter placement”; WO 1995/019193 to Ogle, titled, “Retractable venipuncture catheter needle and receptacle”; WO 1997/005912 to Rohrbough et al, titled, “Retractable venipuncture catheter needle and receptacle”; and WO 1997/021458 to Hwang, titled, “Intravenous catheter with flexible extender and protector against needle tip.”

### SUMMARY OF THE INVENTION

In one aspect, the present invention takes the form of an intravenous catheter insertion device that provides coordinated movement of an access needle, an intravenous catheter and a safety guidewire. The device holds the access needle and the intravenous catheter in a coaxial arrangement for puncturing a vein or other target vessel. A blood flashback chamber provides a visual indication that the tip of the needle is in the lumen of the vein. Upon vein puncture by the access needle, a flexible safety guidewire is advanced through the access needle into the lumen of the vein using an actuation member located on the exterior of the device. With the flexible safety guidewire deployed within the lumen of the vein, the access needle and the intravenous catheter can be safely advanced into the vein until the tip of the intravenous catheter is also within the lumen of the vein. Alternatively, the intravenous catheter can be advanced separately while holding the access needle stationary. Then, the actuation member is actuated to simultaneously withdraw the access needle and the safety guidewire. Preferably, the access needle and the safety guidewire are withdrawn automatically by the action of a spring or other biasing member, leaving only the intravenous catheter in the vein. Once the access needle and the safety guidewire have been withdrawn, the intravenous catheter can be disconnected from the insertion device and connected to a source of intravenous fluid, medication, etc.

In another aspect, the present invention provides an improved method for insertion and placement of an intravenous catheter. The method includes the steps of: puncturing a vein or other target vessel with an access needle arranged coaxially with an intravenous catheter; verifying the location of the access needle tip in the lumen of the vein; advancing a safety guidewire through the access needle into the lumen of the vein, advancing the tip of the intravenous catheter into the vein; and simultaneously withdrawing the access needle and the safety guidewire from the intravenous catheter and from the patient.

Although the invention is described in relation to insertion of an intravenous catheter, the apparatus and methods described herein could readily be adapted for insertion of any catheter or similar device into a vein, artery or other internal body structure.

### BRIEF DESCRIPTION OF THE DRAWINGS

FIG. 1 shows an exploded view of an intravenous catheter insertion device according to the present invention.

FIG. 2 shows an assembly drawing of the intravenous catheter insertion device in an undeployed state, ready for use.

FIG. 3 shows a phantom view of the intravenous catheter insertion device with the safety guidewire advanced.

FIGS. 4A and 4B are detail drawings of a safety guidewire for use with the intravenous catheter insertion device.

FIGS. 5A, 5B and 5C are detail drawings of another safety guidewire for use with the intravenous catheter insertion device.

FIG. 6 shows another embodiment of an intravenous catheter insertion device according to the present invention.

FIGS. 7-9 illustrate a method of intravenous catheter insertion according to the present invention.

### DETAILED DESCRIPTION OF THE INVENTION

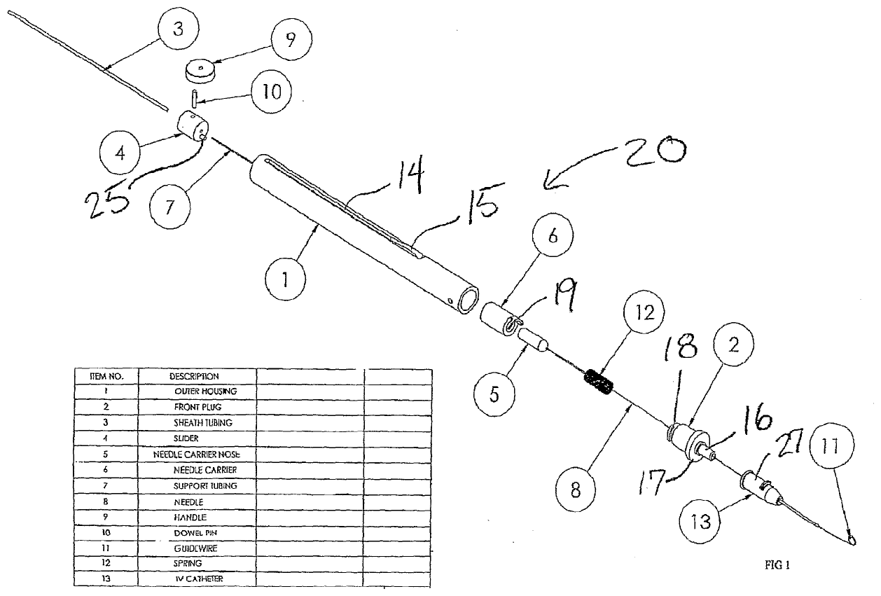

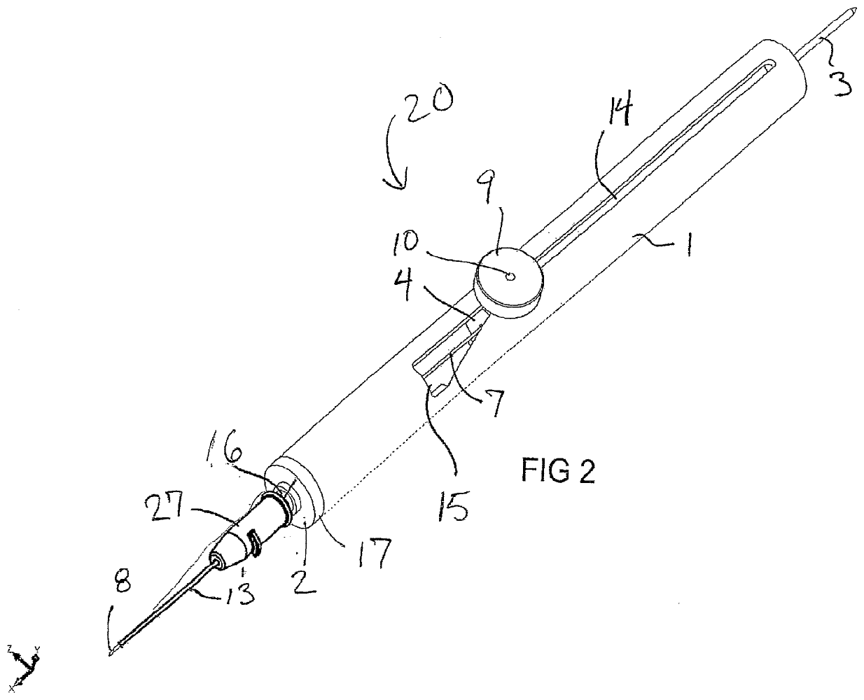

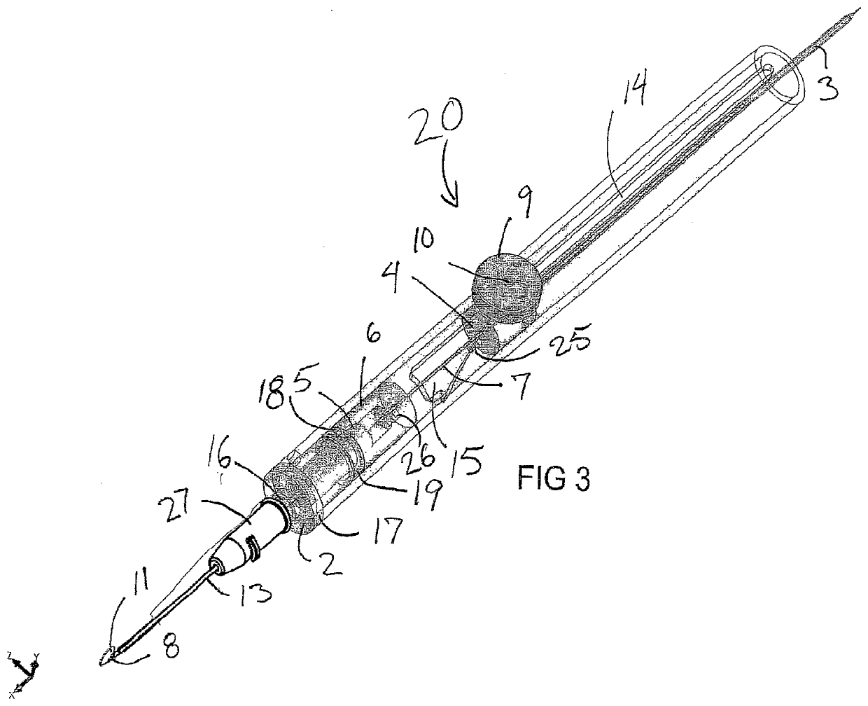

FIG. 1 shows an exploded view of one embodiment of an intravenous catheter insertion device 20 according to the present invention. FIG. 2 shows an assembly drawing of the intravenous catheter insertion device 20 in an undeployed state, ready for use. FIG. 3 shows a phantom view of the intravenous catheter insertion device 20 with the safety guidewire advanced.

The intravenous catheter insertion device 20 includes an outer housing 1. In the example shown, the outer housing 1 is in the form of an elongated hollow cylinder. Other shapes, including an ergonomic handle shape, are possible. The outer housing 1 may be formed from any material suited for use in medical applications. In one embodiment, the outer housing 1 is preferably molded from a rigid, transparent medical grade plastic. Alternatively, the outer housing 1 may be machined from an extruded plastic tube. There is an elongated slot 14 in the outer housing 1 approximately parallel with the axis of the outer housing 1. The slot 14 is sized to accommodate the dowel pin 10 or provide a connection point to the slider 4 to move the slider along the interior of the outer housing 1. The distal end of the slot 14 widens into a triangular cutout 15, as seen in FIGS. 2 and 3. Other shapes of the cut out 15 are possible.

A front plug 2 is sized to fit onto the distal end of the outer housing 1. The front plug 2 is preferably molded, or alternatively machined, from a rigid, transparent medical grade plastic. The front plug 2 is glued, pinned, welded or otherwise fastened to the distal end of the outer housing 1. The distal end of the front plug 2 includes a luer slip fitting 16 or the like. There is a shoulder or flange 17 to mate with the distal end of the outer housing 1. The proximal end of the front plug 2 has an interlocking member 18 that interlocks with a mating interlocking member 19 on the needle carrier 6. In the example shown, the interlocking member 18 is a tab that interlocks with a corresponding spiral pawl or quarter-turn thread interlocking member 19 on the needle carrier 6. Other geometries for the interlocking members 18, 19 are possible.

In the exemplary embodiment of FIGS. 1-3, the geometry of the slot 14 and the triangular cutout 15 are chosen to operate cooperatively with the rotating interlocking members 18, 19. The slot 14 allows the actuator handle 9 to move in a longitudinal direction with respect to the outer housing 1 to advance the safety guidewire 11 distally, while at the same time restricting lateral motion to avoid premature withdrawal of the access needle 8 and the safety guidewire 11. The widening of the slot 14 at the distal end into a triangular cutout 15 allows the actuator handle 9 to be selectively rotated laterally to disengage the rotating interlocking members 18, 19 and release the biasing member 12 to withdrawal of the access needle 8 and the safety guidewire 11 after the safety guidewire 11 has been fully advanced. If a different geometry or different release mechanism is used in place of the rotating interlocking members 18, 19, the geometry of the slot 14 and the triangular cutout 15 may have to be modified to accommodate the release mechanism.

The needle carrier 6 is shaped and sized to fit inside the outer housing 1. In the embodiment shown in FIGS. 1-3, the needle carrier 6 has a cylindrical shape that is sized to have a sliding fit within the cylindrical outer housing 1. Other shapes are possible and generally the needle carrier 6 will be shaped to be compatible with the interior geometry of the outer housing 1. The needle carrier 6 is preferably molded, or alternatively machined, from any material suited for use in a medical environment. In one embodiment, the needle carrier 6 is formed from a rigid, transparent medical grade plastic. A tubular access needle 8 with a sharpened beveled distal end is attached to a needle carrier nose 5, which is in turn attached to the needle carrier 6. The access needle 8 is preferably made from stainless steel hypodermic tubing. A small cavity or blood flashback chamber that communicates with the lumen of the access needle 8 is positioned within the needle carrier 6, between the needle carrier nose 5 and the needle carrier 6. As mentioned above, the distal end of the needle carrier 6 has an interlocking member 19 that is configured to interlock with a mating interlocking member 18 on the proximal end of the front plug 2. In one exemplary embodiment, the interlocking members 18, 19 are adapted to lock and unlock by rotation of the needle carrier 6 with respect to the front plug 2. The interlocking members 18, 19 may also lock and unlock using a bayonet-type fitting. In the example shown, the interlocking member is a spiral pawl interlocking member 19 that interlocks with a corresponding tab interlocking member 18 on the front plug 2. In one embodiment, the interlocking members lock and/or unlock using less than one revolution of the needle carrier 6. In another embodiment, the interlocking members lock and/or unlock using less than one half a revolution of the needle carrier 6. In still another alternative embodiment, the interlocking members lock and/or unlock using less than one quarter revolution of the needle carrier 6. Other geometries for the interlocking members are possible.

A biasing member 12 is configured to fit between the needle carrier 6 and the front plug 2 to urge them apart. The force of the biasing member 12 is resisted by the interlocking members 18, 19 when the needle carrier 6 and the front plug 2 are locked together. In one embodiment, the biasing member 12 is a spring. Note that in FIG. 1 the biasing member or compression spring 12 is shown in a compressed condition as it would be in the assembled intravenous catheter insertion device 20 in an undeployed condition.

In an alternate embodiment, the interlocking members 18, 19 may be replaced by two members that are bonded together with a breakable bond or a single member with a breakable link. The member or members would be configured to constrain the biasing member 12 until it is desired to withdraw the access needle 8 and safety guidewire 11, at which time, the actuator would break the bond or link to release the biasing member 12. This configuration would make the device 20 more resistant to remanufacturing or reuse.

A tubular intravenous catheter 13, such as an ANGIOCATH, fits coaxially around the access needle 8. Preferably, the intravenous catheter 13 has a close fit with the access needle 8 and a tapered distal end to minimize any step between the access needle 8 and the intravenous catheter 13 as they are inserted through the wall of a vein. There is a luer fitting 27 or the like on the proximal end of the intravenous catheter 13 that fits onto the luer slip fitting 16 on the distal end of the front plug 2 with a slight interference fit to hold the intravenous catheter 13 in place. Alternative configurations of the device may use a luer lock or other locking mechanism to attach the intravenous catheter 13 to the front plug 2.

A slider 4 is generally cylindrical in shape and sized for a sliding fit inside the cylindrical outer housing 1. Other shapes for the slider 4 are possible depending on the interior geometry of the outer housing 1. The slider 4 is preferably molded, or alternatively machined, from any suitable medical grade material. For example, the slider may be formed from a rigid medical grade plastic. A handle 9 or actuating member attaches to the slider 4 with a dowel pin 10 or other attachment member that extends through the slot 14 in the outer housing 1. The slider 4 fits into the outer housing 1 proximal to the needle carrier 6. A pin 25 extends from the distal surface of the slider 4 and is configured to reversibly engage with a hole, step, boss or similar mating feature 26 on the proximal end of the needle carrier 6. When pin 25 is coupled to the mating feature 26 during the appropriate step of the intravenous catheter insertion and placement procedure, rotation of the slider 4 is transferred to the needle carrier 6 to facilitate engagement and or disengagement of the interlocking members 18, 19. Pin 25 and feature 26 are merely illustrative. Pin 25 may be replaced with a female feature while a mating male feature may be placed on the proximal face of the needle carrier 6. Additionally, the mating features 25, 26 are aligned relative to the elongated slot and the sliding movement of the slider 4 so that distal movement of the slider 4 will engage the mating features 25, 26. Optionally, the device 20 may be configured so that the connection between the slider 4 and needle carrier 6 happens irreversibly when the device 20 is actuated.

As best seen in FIG. 3, a safety guidewire 11 is attached, directly or indirectly, to the slider 4 so that it can be advanced and retracted with the handle 9 attached to the slider 4. In a preferred embodiment, the safety guidewire 11 is constructed of superelastic Nickel-Titanium alloy (Nitinol) wire. Because this type of wire is extremely flexible, it is advantageous to have the safety guidewire 11 enclosed along most of its length to avoid bowing or buckling while advancing the safety guidewire 11. For this reason, the example shown includes a support tubing 7 that is attached to the proximal end of the needle carrier 6. The safety guidewire 11 extends through the internal lumen of a sheath tubing 3 and the proximal end of the safety guidewire 11 is attached at the proximal end of the sheath tubing 3. The distal end of the sheath tubing 3 is in turn attached to the slider 4, indirectly attaching the safety guidewire 11 to the slider 4. The support tubing 7 has a sliding fit inside the sheath tubing 3 so that the two parts telescope together as the slider 4 is advanced in the distal direction. The telescoping action of the support tubing 7 and the sheath tubing 3 provides a variable length support for the proximal portion of the safety guidewire 11 to prevent bowing or buckling of the safety guidewire 11 as it is advanced. The support tubing 7 and the sheath tubing 3 are preferably made from stainless steel hypodermic tubing, however any suitable medical grade plastic material may also be used. In other embodiments, such as those using a larger diameter or stiffer guidewire, the telescoping support tubes may not be necessary, and the proximal end of the safety guidewire 11 may be attached directly to the slider 4.

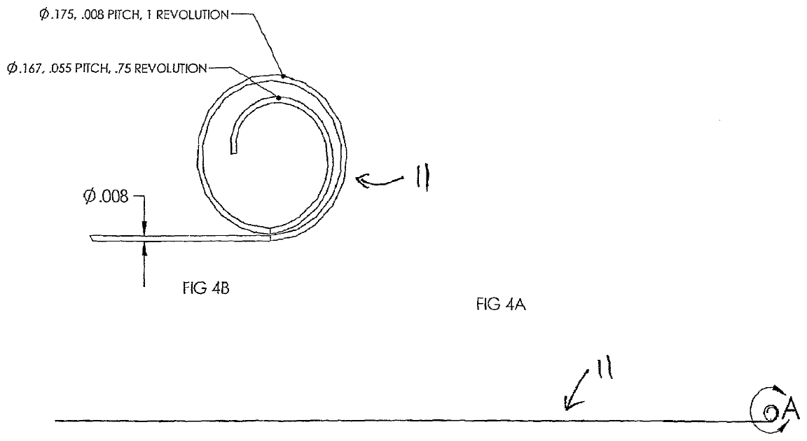

FIGS. 4A and 4B are detail drawings of a safety guidewire 11 for use with the intravenous catheter insertion device 20. The safety guidewire 11 is preferably constructed of superelastic Nickel-Titanium alloy wire approximately 0.004-0.012 inches in diameter and most preferably approximately 0.008 inches in diameter. As shown in FIG. 4B, the distal end of the safety guidewire 11 is preformed into a tightly wound spiral with an outer diameter smaller than the internal diameter of the target vessel into which it will be inserted. The spiral tip acts as a safety bumper on the guidewire to avoid puncturing or damaging the inside of target vessels. The coiled guidewire tip is particularly useful in protecting fragile or delicate veins. Due to the extreme flexibility of the Nickel-Titanium alloy wire, the spiral distal curve can straighten out when the safety guidewire 11 is withdrawn into the access needle 8 and completely recover into the spiral configuration without plastic deformation when the safety guidewire 11 is advanced out of the access needle 8. In the example shown, the distal end of the safety guidewire 11 has a first, small diameter coil of approximately 0.167 inches in diameter for approximately 0.75 revolutions and a second, larger diameter coil of approximately 0.175 inches in diameter for approximately 1 revolution. The first and second coils are preferably approximately coplanar with one another and preferably approximately coplanar with the straight proximal portion of the guidewire 11 also. Other configurations of the safety guidewire 11 may include: multi-planar, single coil, full radius on the end, and/or a balled end with diameter less than the diameter of the needle.

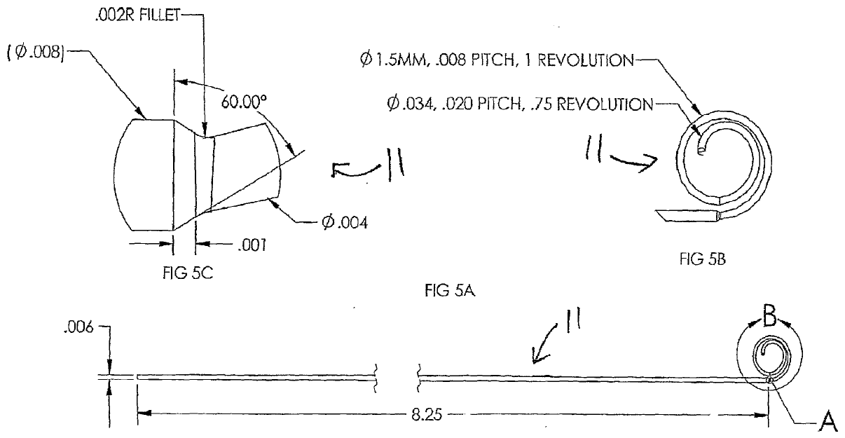

FIGS. 5A, 5B and 5C are detail drawings of another safety guidewire 11 for use with the intravenous catheter insertion device 20. In this embodiment, a distal portion of an approximately 0.008 inch diameter Nickel-Titanium alloy wire has been tapered by grinding, stretching, etc., to a diameter of approximately 0.004 inches to make it more flexible and to allow it to be formed into a smaller diameter spiral for use in smaller diameter veins. The spiral curve of the guidewire tip will preferably have an outer diameter smaller than the inner diameter of the target vessel. In the example shown, the spiral curve has a first, small diameter coil of approximately 0.034 inches in diameter for approximately 0.75 revolutions and a second, larger diameter coil of approximately 0.059 inches in diameter for approximately 1 revolution. The first and second coils are preferably approximately coplanar with one another and preferably approximately coplanar with the straight proximal portion of the guidewire 11 also.

Other sizes and geometries of safety guidewire 11 are also possible.

To assemble the intravenous catheter insertion device 20 shown in FIGS. 1-3, the access needle 8 is bonded flush with the proximal face of the needle carrier nose 5, which is in turn bonded into the needle carrier 6. The support tubing 3 is placed into the distal hole in the needle carrier 6, and bonded flush with the proximal face of the blood flashback chamber. The formed safety guidewire 11 is advanced through the lumen of the access needle 8 and support tubing 7 until the coiled section of the safety guidewire 11 meets the access needle 8 bevel. The sheath tubing 3 is slid through the slider 4, and bonded when flush with the distal face. The assembly of the sheath tubing 3 and slider 4 are advanced over the safety guidewire 11. When the safety guidewire 11 is flush with the proximal end of the sheath tubing 3, the two are bonded. The spring 12 is compressed on the needle carrier nose 5, advanced into the front plug 2 and the interlocking members 18, 19 of the front plug 2 and needle carrier 6 are engaged. This assembly of components is placed into the outer housing 1 and advanced until the front plug 2 is flush with the outer housing 1, and then the front plug 2 is rotated for proper alignment. The front plug 2 is then bonded to the outer housing 1. The dowel pin 10 and handle 9 are pressed together with the slider 4. The handle 9 is slid proximally to withdraw the safety guidewire 11 into the access needle 8, thereby straightening out the spiral distal curve. An intravenous catheter 13 is then mounted coaxially around the access needle 8. Optionally, the intravenous catheter 13 insertion device may be provided with a needle cover or other protective packaging. The assembled intravenous catheter insertion device 20, including the intravenous catheter 13, is then packaged, labeled and sterilized.

The preceding assembly description is provided to illustrate one example of a process for manufacturing an embodiment of the intravenous catheter insertion device 20 and also so that the interrelationship of the various components will be understood. Modifications and variations of this description are expected depending upon specific selected assembly or manufacturing techniques. For example, components that are bonded may be redesigned to be formed from a single integrated piece and the like. The manufacturing process can be modified and adapted for assembling other embodiments of the intravenous catheter insertion device 20.

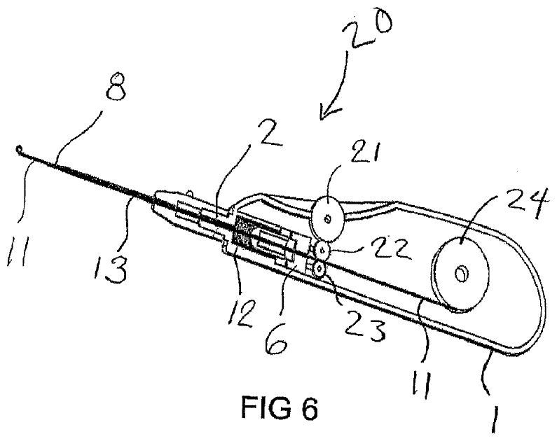

FIG. 6 shows an interior view of another embodiment of an intravenous catheter insertion device 20 according to the present invention. This embodiment is similar in many respects to the intravenous catheter insertion device 20 of FIGS. 1-3. The intravenous catheter insertion device 20 includes an outer housing 1, front plug 2, which may optionally be molded integrally with the outer housing 1, a needle 8 attached to a needle carrier 6, a safety guidewire 11, spring 12 and intravenous catheter 13. However, the functions of the handle 9 and the slider 4 have been replaced by a thumbwheel 21 that engages a pair of friction wheels 22, 23, which are in contact with the safety guidewire 11. Likewise, the functions of the sheath tubing 3 and the support tubing 7 have been replaced by a guidewire spool 24. These features allow the intravenous catheter insertion device 20 to be constructed in a more compact configuration. In use, the safety guidewire 11 is advanced by turning the thumbwheel 21. A lateral movement of the thumbwheel 21 disengages the needle carrier 6 from the front plug 2, allowing the biasing member 12 to expand, thereby retracting the needle 8 and the safety guidewire 11 into the outer housing 1. Alternatively, a separate button, lever or other actuation member can be provided to actuate the withdrawal of the needle 8 and the safety guidewire 11. The guidewire spool 24 may optionally include a rotary spring or similar mechanism (not shown) to assist in the retraction of the safety guidewire 11 into the outer housing 1.

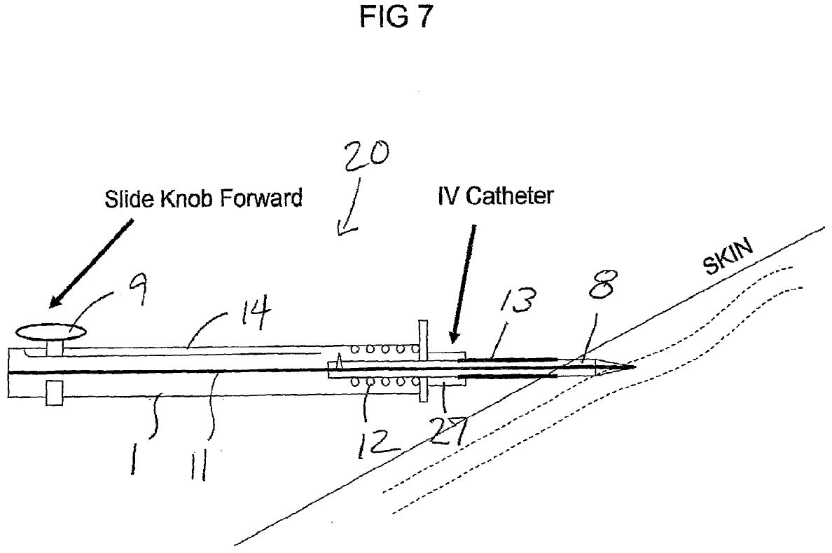

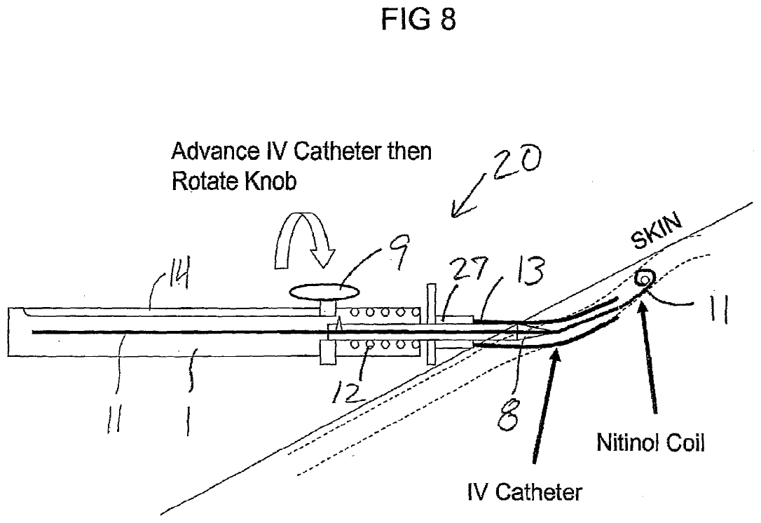

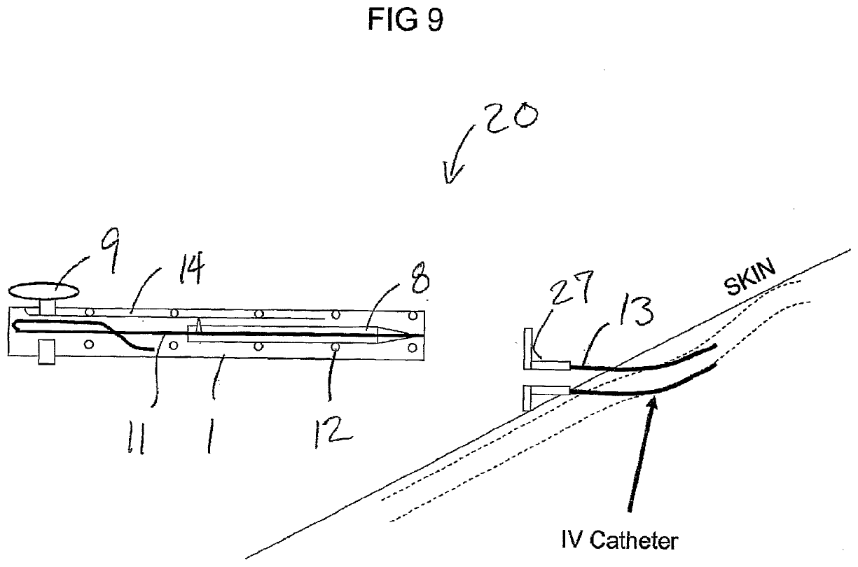

FIGS. 7-9 illustrate a method of inserting an intravenous catheter using an intravenous catheter insertion device 20, such as those described in FIGS. 1-3 or FIG. 6. The intravenous catheter insertion device 20 is a single-use, non-reusable device supplied to the physician or medical practitioner sterile in a ready-to-use, undeployed condition as shown in FIG. 2. In use, the physician uses the outer housing 1 as a handle to manipulate the intravenous catheter insertion device 20. With the device in the undeployed condition, the access needle 8 is used to puncture a vein, as shown in FIG. 7. When venous blood is observed in the blood flashback chamber, the distal tip of the access needle 8 is the lumen of the vein. The physician can then advance the handle 9 in the distal direction to extend the safety guidewire 11 out of the access needle 8 into the lumen of the vein. The distal portion of the safety guidewire 11 assumes its spiral configuration to act as a safety bumper to prevent accidental puncture of the far wall of the vein or other damage to the vein. With the safety guidewire 11 thus deployed, the physician can safely continue advancing the intravenous catheter insertion device 20 until the distal tip of the intravenous catheter 13 is in the lumen of the vein. Once the intravenous catheter 13 is inserted far enough into the vein, the physician rotates the handle 9 that rotates the slider 4, which in turn rotates the needle carrier 6 and disengages the interlocking member 18 of the needle carrier 6 from the mating interlocking member 19 on the front plug 2. (In the exemplary embodiment described above, the handle moves in a counterclockwise direction as allowed by the triangular cutout 15 at the distal end of the slot 14 in the outer housing 1. Additional structural features of the actuator mechanism are shown in more detail in FIGS. 1-3.) When the handle 9 is released, the biasing element (here a compression spring 12) urges the needle carrier 6 and the slider 4 in the proximal direction, thus simultaneously withdrawing the access needle 8 and the safety guidewire 11 into the outer housing 1, leaving only the intravenous catheter 13 in the lumen of the vein. FIG. 8 shows the access needle 8 and the safety guidewire 11 withdrawing into the outer housing 1. The shape of the triangular cutout 15 allows the handle 9 to make a smooth transition into the elongated slot 14 as it moves proximally under the influence of the biasing element 12. Finally, the intravenous catheter 13 is disengaged from the luer slip 16 fitting on the distal end of the front plug 2, as shown in FIG. 9, and a source of intravenous fluid, a syringe or other device is attached to the luer fitting 27 of the intravenous catheter 13.

While it is desirable for the intravenous catheter insertion device 20 to withdraw the access needle 8 and the safety guidewire 11 simultaneously, the actuator mechanism could also be modified to withdraw the access needle 8 and the safety guidewire 11 sequentially. For example, the actuator mechanism could withdraw the access needle 8 first and then, after a slight delay, withdraw the safety guidewire 11.

Alternatively, the actuator mechanism could be modified to require two separate motions of one actuator member or selective movements of two separate actuator members to withdraw the access needle 8 and the safety guidewire 11 selectively.

In an alternative embodiment of the intravenous catheter insertion device 20, the compression spring 12 may be omitted from the actuator mechanism, thus allowing the access needle 8 and the safety guidewire 11 to be withdrawn manually using the handle 9. Once the intravenous catheter 13 has been inserted into the patient's vein, the handle 9 is rotated laterally to disengage the needle carrier 6 from the front plug 2, then the handle 9 is moved proximally along the slot 14 to withdraw the access needle 8 and the safety guidewire 11 into the outer housing 1.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. For example, all dimensions and materials included in the specification or drawings are intended only as examples of presently preferred embodiments and are not intended to limit the scope of the invention.

What is claimed is:

1. A method for accessing a blood vessel, comprising:
preparing a catheter insertion device for introduction into the blood vessel, the catheter insertion device comprising:
a housing;
a needle having a proximal end in the housing;
a catheter positioned over the needle, the catheter including a catheter hub;
a guidewire disposed in a lumen of the needle; and
a thumbwheel rotatably fastened at a front end of the housing and accessible through an opening in the housing, the guidewire coupled to the thumbwheel, wherein:
an axis of rotation of the thumbwheel is perpendicular to the guidewire; and
rotation of the thumbwheel advances the guidewire through the lumen of the needle;
inserting a tip of the needle and a distal end of the catheter into a blood vessel of the patient;
advancing the guidewire out of the lumen of the needle into the blood vessel without moving the catheter and the needle by holding the housing and rotating the thumbwheel;
furthering the distal end of the catheter into the blood vessel over the guidewire; and
removing the needle and the guidewire from the catheter.

2. The method according to claim 1, wherein the catheter insertion device further comprises a blood flashback chamber, further comprising observing blood in the blood flashback chamber after inserting the needle and the catheter into the blood vessel prior to rotating the thumbwheel.

3. The method according to claim 1, wherein the thumbwheel engages a friction wheel in the housing, and wherein rotating the thumbwheel rotates the friction wheel to advance the guidewire.

4. The method according to claim 1, wherein the needle is attached to a needle carrier.

5. The method according to claim 4, wherein the catheter insertion device further comprises an actuator mechanism including a biasing member positioned between a distal end of the housing and the needle carrier, and wherein activating the actuator mechanism releases the biasing member.

6. The method according to claim 5, wherein activating the actuator mechanism to release the biasing member simultaneously retracts the needle and the guidewire into the housing.

7. The method according to claim 5, wherein activating the actuator mechanism comprises moving the thumbwheel laterally to disengage the needle carrier from a front plug in the housing.

8. The method according to claim 1, wherein the guidewire has a proximal portion having a first diameter and a distal portion having a second diameter less than the first diameter, wherein a distal end of the guidewire has a straight configuration in the lumen of the needle and a coiled configuration outside of the lumen of the needle, and wherein advancing the guidewire out of the lumen of the needle transitions the distal end of the guidewire from the straight configuration to the coiled configuration.

9. The method according to claim 1, wherein a distal end of the guidewire has a coiled configuration including a first coil in a first coil plane and a second coil in a second coil plane different from the first coil plane, and wherein advancing the distal end of the guidewire out of the lumen of the needle transitions the distal end of the guidewire from a straight configuration to the coiled configuration.

10. The method according to claim 9, wherein advancing the guidewire out of the lumen of the needle transitions the distal end of the guidewire to the coiled configuration without plastic deformation of the guidewire.

11. The method according to claim 1, wherein:
a distal end of the guidewire has a coiled configuration, comprising:
a first coil extending from a straight portion of the guidewire, the first coil lying substantially in a first coil plane; and
a second coil extending from the first coil, the second coil lying substantially in a second coil plane parallel to the first coil plane, the second coil having a diameter less than or equal to a diameter of the first coil, wherein a line orthogonal to the first coil plane and the second coil plane is substantially orthogonal to the straight portion of the guidewire, and
advancing the distal end of the guidewire out of the lumen of the needle transitions the distal end of the guidewire from a straight configuration to the coiled configuration.

12. The method according to claim 11, wherein a distal tip of the guidewire meets a bevel of the needle in a fully withdrawn position, and wherein furthering the distal end of the catheter over the guidewire into the blood vessel comprises furthering the distal end of the catheter over the distal end of the guidewire in the coiled configuration.

13. The method according to claim 1, wherein the catheter hub is coupled to a distal end of the housing via an interference fit, wherein moving the distal end of the catheter over the guidewire into the blood vessel comprises uncoupling the catheter hub from the distal end of the housing.

14. The method according to claim 1, wherein the catheter hub is coupled to a member extending from a distal end of the housing, and wherein furthering the catheter over the distal end of the guidewire into the blood vessel comprises uncoupling the catheter hub from the member.
